(19) United States
(12) Patent Application Publication
(10) Pub. No.: US 2025/0241889 A1
(43) Pub. Date: Jul. 31, 2025

(54) FORTIFIED WATER

(71) Applicant: Lovaness Limited, Inverness (GB)

(72) Inventors: Elena Lurie-Luke, Inverness (GB); Vladimir Bassonov, Inverness (GB)

(21) Appl. No.: 18/704,777

(22) PCT Filed: Oct. 26, 2022
(86) PCT No.: PCT/GB2022/052724

§ 371 Date: Apr. 25, 2024

(30) Foreign Application Priority Data
Oct. 27, 2021 (GB) 2115456.2

(51) Int. Cl. A61K 31/352; A23L 2/52; A61K 9/00; A61K 31/353; A61K 31/355; A61K 31/375; A61K 47/10; A61K 47/12; A61K 47/26; A61K 47/40; C02F 1/68; C02F 103/02

(52) U.S. Cl. CPC A61K 31/352; A23L 2/52; A61K 9/0095; A61K 31/353; A61K 31/355; A61K 31/375; A61K 47/10; A61K 47/12; A61K 47/26; A61K 47/40; C02F 1/68; C02F 2103/02

(57) ABSTRACT

The present invention is directed to fortified drinking water compositions containing flavonoid compounds, to packaged products containing such fortified drinking water compositions and to methods for preparing such products.

### FIELD OF THE INVENTION

The present invention is directed to drinking water compositions supplemented with flavonoid compounds. The invention also relates to packaged products containing such fortified drinking water compositions and to methods for preparing such products.

### BACKGROUND OF THE INVENTION

Taxifolin, also known as dihydroquercetin, is one example of the broad family of potent natural flavonoid antioxidants. It is present in vinegars aged in cherry wood and, like flavonoids in general, can be found in an array of plant-based foods like fruit, vegetables and cocoa.

Many flavonoids, including taxifolin, are used as an additive in food products, not least owing to their health benefit as antioxidant and anti-inflammatory agents. For example, flavonoids in general, and in particular taxifolin, have been shown to protect against a vast array of conditions, including liver and/or kidney damage^{1,2,3,4,5}, hypertension⁶, vascular damage^{7,8}, cardiac hypertrophy⁹, and elevated blood pressure and/or cholesterol levels^{10,11}.

In light of such health benefits, the Russian State Food Research Institute have calculated a recommended dietary allowance (RDA), i.e. the average daily dietary intake level that suffices to meet the nutrient requirements of the general population, for flavonoids such as taxifolin of 25 mg per day¹⁵. However, at present, the population as a whole often fall well short of this level of flavonoid intake from diet alone. Further, whilst this shortfall may be addressed by the use of mineral supplements, this is far from optimal not least because adherence to supplement intake regimens requires modification of consumer behaviour and habits. For instance, RU2277833C1 discloses a mineral drink water comprising dihydroquercertin, amongst other additives such as flavourings and vitamin C. Similarly, RU2699548C1 discloses a water-based health drink comprising taxifolin amongst other additives (e.g. colouring, flavouring). RU2498801C1 discloses storage stable compositions comprising water-soluble taxifolin but does not disclose taxifolin-fortified drinking water as such. Other prior art discloses fortified drinks comprising dietary flavonoids, such as taxifolin added to alcoholic and non-alcoholic beverages¹⁶, or benefits of dietary flavanols provided in the form of cocoa-based drinks¹⁷. However, it would be advantageous to provide consumers with a drinking water composition fortified with one or more flavonoids that meets the RDA without artificial additives, such as colorants and/or flavourings, in order to maximise the health benefits of the drinking water.

It is, therefore, an object of the present invention to design a new, convenient form of flavonoids such as, for example, taxifolin that will drive consumption to RDA levels, and hence provide health benefits to the population, without the need to rely on mineral supplements and associated changes in consumer behaviour. Further, it would be advantageous if flavonoids, such as taxifolin, could be administered in a convenient form, optionally alongside additional vitamins and minerals, by incorporation into a daily routine of drinking water. Further still, in order to be well accepted, the incorporation of flavonoid compounds into drinking water should not negatively impact on appearance and/or flavour.

### STATEMENTS OF INVENTION

The present invention, in its various aspects, is as set out in the accompanying claims.

According to a first aspect of the invention, there is provided a drinking water composition having a pH of from 4 to 9.5, wherein said composition is fortified with one or more flavonoid compound in a total amount of at least 0.025 mg/ml, wherein said composition contains no additional natural or artificial colorants, flavours or preservatives. For the avoidance of doubt, the drinking water composition of the present disclosure may comprise one flavonoid compound or may comprise a combination of different types of flavonoid compounds provided herein.

In preferred embodiments, said one or more flavonoid compound is selected from the group consisting of: flavan-3-ols (also known as flavanols), anthocyanidins, flavanones, flavones, flavonols, flavanonols, and/or glycosides thereof. In some embodiments, said one or more flavonoid compound is selected from the group consisting of: flavan-3-ols (also known as flavanols), flavanones, flavones, flavonols, flavanonols, and/or glycosides thereof. In other preferred embodiments, said one or more flavonoid compound is selected from the ground consisting of: flavan-3-ols, flavonols, flavanonols, and/or glycosides thereof. More preferably, said one or more flavonoid compound(s) is a flavonol, a flavanonol and/or flavan-3-ol. In particularly preferred embodiments, the flavanonol compound is taxifolin. In an alternative preferred embodiment, said flavonoid compound is a flavan-3-ol and/or a glycoside thereof. In some embodiments, the flavan-3-ol compound is catechin, epicatechin and/or gallocatechin. In some embodiments, the flavan-3-ol compound is catechin. In some embodiments, the flavan-3-ol compound is epicatechin. In some embodiments, the flavan-3-ol compound is gallocatechin.

As will be readily appreciated, the term “flavonoid” refers to compounds having the general structure of a 15-carbon skeleton consisting of two phenyl rigs (ring A and ring B) and a heterocyclic ring (ring C), which may be saturated (as shown below) or unsaturated and includes an embedded oxygen. Such a structure, which can be abbreviated C6-C3-C6, is shown below.

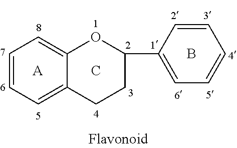

As will be readily appreciated, the terms “flavan-3-ol” and “flavanol” refer to compounds having a 2-phenyl-3,4-dihydro-2H-chromen-3-ol skeleton as shown below.

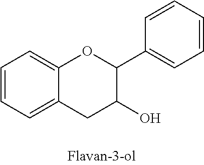 [SMILES: OC1Cc2ccccc2OC1c1ccccc1]

As will be readily appreciated, the term “anthocyanidin” refers to compounds having the general structure shown below, wherein R³ and R^{4′} are both OH. Glycosylated anthocyanidins, i.e. anthocyanins, are included within the scope of such compounds.

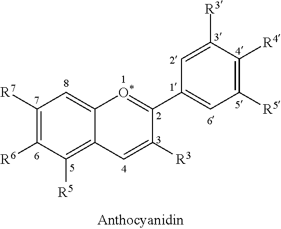

As will be readily appreciated, the term “flavanone” refers to compounds having the general backbone structure shown below.

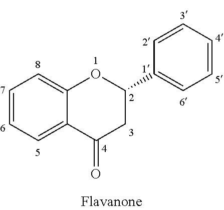 [SMILES: O=C1C[C@@H](c2ccccc2)Oc2ccccc21]

As will be readily appreciated, the term “flavone” refers to compounds comprising a 2-phenyl-3,4-dihydro-2H-chromen-3-ol backbone as shown below.

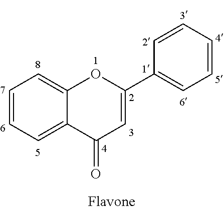 [SMILES: O=c1cc(-c2ccccc2)oc2ccccc12]

As will be readily appreciated, the term “flavonol” refers to compounds comprising a 3-hydroxyflavone (IUPAC name: 3-hydroxy-2-phenylchromen-4-one) backbone as shown below.

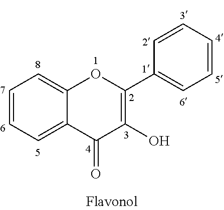 [SMILES: O=c1c(O)c(-c2ccccc2)oc2ccccc12]

In the present specification, the term “catechin” refers to (2R,3S)-2-(3,4-Dihydroxyphenyl)-3,4-dihydro-2H-chromene-3,5,7-triol, the structure of which is shown below.

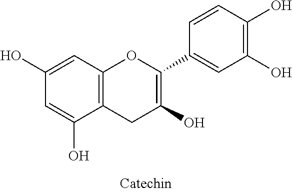

In the present specification, the term “taxifolin” refers to (2R,3R)-2-(3,4-dhydroxyphenyl)-3,5,7-trihydroxy-2,3-dihydro-4H-1-benzopyrane-4-one (also known as 5,7,3′,4′-flavan-on-ol) and dihydroquercetin), the structure of which is shown below.

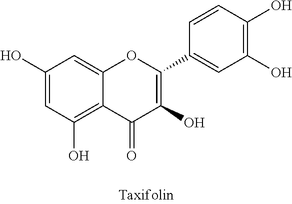

In preferred embodiments, the flavonoid compound, e.g. catechin and/or taxifolin, is provided in the drinking water composition in the form of a stabilized water soluble flavonoid solution. Many examples of such stabilized solutions are known and readily available. Other such stabilized solutions can readily be readily designed by a person of skill in the art using common general knowledge.

Exemplary stabilized water soluble taxifolin solutions, which are disclosed in Russian Patent No. RU 2 498 801 C1, comprise taxifolin; 2-hydroxypropyl-beta-cyclodextrin; one or more of propylene glycol, glycerol and PEG 400; one or more organic acid or a water-soluble salt thereof; and one or more carbohydrates selected from monosaccharides, disaccharides and maltodextrins in the following amounts with distilled water to balance:
Taxifolin: 0.1 to 30 wt. %
2-hydroxypropyl-beta-cyclodextrin: 0.01-25 wt. %
propylene glycol, glycerin and/or PEG-400:1-70 wt. %
organic acid/salt: 0.1-3 wt. %
carbohydrate: 0.01-30 wt. %.

In such stabilized taxifolin compositions, said organic acid is preferably selected from the group consisting of succinic acid, malic acid, ascorbic acid, isoascorbic acid, citric acid, lactic acid, fumaric acid, tartaric acid, acetic acid, sorbic acid, benzoic acid, and combinations thereof.

Similarly, said carbohydrate is preferably selected from the group consisting of glucose, fructose, sorbitol, xylitol, galactose, mannose, sucrose, lactose, maltose, isomaltose, lactulose, and combinations thereof.

In preferred embodiments, the composition comprises the flavonoid compound(s) in a total amount from 0.025 mg/ml to 0.5 mg/ml. More preferably, the flavonoid compound(s) are present in an amount of at least 0.05 mg/ml, still more preferably at least 0.075 mg/ml, still more preferably 0.125 mg/ml, and more preferably still at least 0.25 mg/ml. Similarly, the flavonoid compound(s) are more preferably present in an amount of 0.5 mg/ml or less, 0.25 mg/ml or less, more preferably 0.125 mg/ml or less, and still more preferably 0.075 mg/ml or less. In one embodiment, the flavonoid compound(s) are more preferably present in an amount of 2 mg/ml or less. In one embodiment, the flavonoid compound(s) are more preferably present in an amount of 1.5 mg/ml or less. In one embodiment, the flavonoid compound(s) are more preferably present in an amount of 1 mg/ml or less. In one embodiment, the flavonoid compound(s) are more preferably present in an amount of 0.75 mg/ml or less. In a preferred embodiment, the flavonoid compound(s) are more preferably present in an amount of 0.5 mg/ml or less. In a preferred embodiment, the flavonoid compound(s) are more preferably present in an amount of 0.25 mg/ml or less.

In an exemplary embodiment, the drinking water composition comprises flavonoid compound(s), preferably taxifolin, catechin, epicatechin and/or gallocatechin, in a total amount of about 0.05 mg/ml or more. In one embodiment, the drinking water composition comprises flavonoid compound(s), preferably taxifolin, in a total amount of about 0.05 mg/ml or more. In an alternative exemplary embodiment, the drinking water composition comprises flavonoid compound(s), preferably catechin, epicatechin and/or gallocatechin, in a total amount of about 0.05 mg/ml or more. In an alternative exemplary embodiment, the drinking water composition comprises flavonoid compound(s), preferably catechin, in a total amount of about 0.05 mg/ml or more. In an alternative exemplary embodiment, the drinking water composition comprises flavonoid compound(s), preferably epicatechin, in a total amount of about 0.05 mg/ml or more. In an alternative exemplary embodiment, the drinking water composition comprises flavonoid compound(s), preferably gallocatechin, in a total amount of about 0.05 mg/ml or more. In some embodiments, any of the flavonoids disclosed herein may be provided at a concentration of 0.025 mg/ml, 0.075 mg/ml, 0.01 mg/ml, 0.02 mg/ml, 0.03 mg/ml, 0.04 mg/ml, 0.06 mg/ml, 0.07 mg/ml, 0.08 mg/ml, 0.09 mg/ml, 0.1 mg/ml or more. The inventors have surprisingly found that fortifying water with exemplary flavonoids, in particular taxifolin, catechin, epicatechin and/or gallocatechin, to this level, it is possible to provide 100% of flavonoid RDA in a single (e.g. 0.5 L) serving without any impact on appearance, odour and/or taste. In one embodiment, the concentration of the flavonoid compound(s) is such that 100% of RDA can be met, for example, in a single serving comprising 250 ml, 330 ml, 500 ml and/or 700 ml of drinking water, without any impact on appearance, odour and/or taste. It would be appreciated by the skilled person in the art when the drinking water comprising different types of flavonoids, each flavonoid may be constituted at different concentrations. In one embodiment, the drinking water comprising flavan-3-ol may be provided at four-fold, three-fold or two-fold greater concentration than flavanonol.

The drinking water compositions of the present invention may be fortified only with said flavonoid compound(s), or optionally may be further fortified with additional additives, provided that the inclusion of such additional components does not impact on appearance and/or taste of the drinking water composition. In a preferred embodiment, the drinking water compositions of the present disclosure may be fortified with one or more flavonoid compound(s), or optionally may be further fortified with additional additives, without negatively affecting the organoleptic properties of the water. Organoleptic properties may include perception of taste, sight and/or smell, for example. In a preferred embodiment, the drinking water composition of the present disclosure remains colourless to the naked eye, such that when compared with unfortified water or pure water, there is no significant difference in colour and/or opacity. In addition, or alternatively, the drinking water compositions of the present disclosure may be fortified with one or more flavonoid compound(s), or optionally may be further fortified with additional additives, without negatively affecting the taste and/or odour of the water. For example, the drinking water composition may have an acceptable taste and/or odour compared to unfortified water or pure water, such that the drinking water composition of the present disclosure has an acceptable level of taste, bitterness and/or tartness. In one embodiment, the drinking water composition has no discernible taste and/or odour compared to unfortified water or pure water.

In particularly preferred embodiments, the drinking water composition further comprises one or more additive selected from vitamin C and vitamin E. The inclusion of such additional additives should not have a deleterious effect on appearance and/or taste of the composition. Moreover, both of these compounds have been shown to interact with the flavonoid(s) in a synergistic manner^{12,13,14}, thereby augmenting the antioxidant effect.

In some embodiments, the drinking water composition comprises mineral water, preferably spring water or well water.

As noted above, the inclusion of flavonoid compounds, such as taxifolin, catechin, epicatechin and/or gallocatechin, in the drinking water compositions of the present invention does not negatively impact on the taste and/or appearance of the water. Therefore, in preferred embodiments, the drinking water composition comprises no additional natural or artificial colorants, flavours or preservatives which may have been required to mask any discolouration and/or flavouring of the water.

According to a second aspect of the invention, there is provided a packaged drinking water product comprising a composition according to the first aspect of the invention disposed within an oxygen impermeable container. In these aspects of the invention, the flavonoid fortified water compositions have enhanced stability and shelf life.

The oxygen impermeable container may be provided in the form of a can or a bottle, although other containers, e.g. a soft pouch, could be used.

The packaged drinking water products of the second aspect of the invention may be prepared by the methods described below.

In particular, a flavonoid fortified drinking water composition may be prepared by adding one or more flavonoid compounds and, optionally, any further additives to drinking water. Then, one or more oxygen impermeable containers are filed with said flavonoid fortified drinking water composition before said one or more containers are sealed. This method itself represents a third aspect of the invention.

As will be readily appreciated, additional steps may be included in the above method. For example, in order to increase shelf life, the method may further comprise the step of purifying and/or sterilizing the flavonoid fortified drinking water composition, preferably prior to sealing the container. Examples of such purification and/or sterilization steps include, but are not limited to, disinfection (including UV disinfection or preferably pasteurisation), filtration, decanting, CO₂ removal, and activated alumina treatment.

An exemplary method of disinfecting the flavonoid fortified drinking water of the present disclosure comprises pasteurisation. Water may be purified by heat treatment without boiling, through pasteurisation. Pasteurisation does not sterilise water but can reduce pathogen loads by many orders of magnitude, depending on the temperature, contact time, and heat resistance of the pathogen. There are three main type of pasteurisation regimes: long, short and flash. A choice of pasteurisation regime depends on a compound structure. Low Temperature Long Pasteurisation (LTL) at a temperature of 63-65° C. for 30-40 minutes. High Temperature Short Pasteurisation (HTS): at a temperature of 82-85° C. for 0.5-1 minute. Flash Pasteurisation: at a temperature of 92-98° C. for several seconds. As it will be appreciated by the skilled person in the art, any of the abovementioned conditions or other conditions known in the art may be used to purifying and/or sterilizing the flavonoid fortified drinking water composition of the present disclosure. In one embodiment, the fortified water of the present disclosure comprising any of the flavonoids disclosed herein may be disinfected using pasteurisation.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The Invention will now be described by way of example only with reference to the Examples below and to the following Figures wherein:

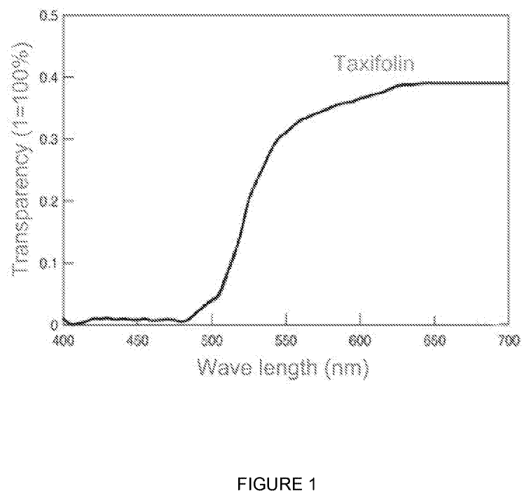

FIG. 1: Transmission spectrum of the base line taxifolin solution (Solution 1:3000 mg/1000 ml)

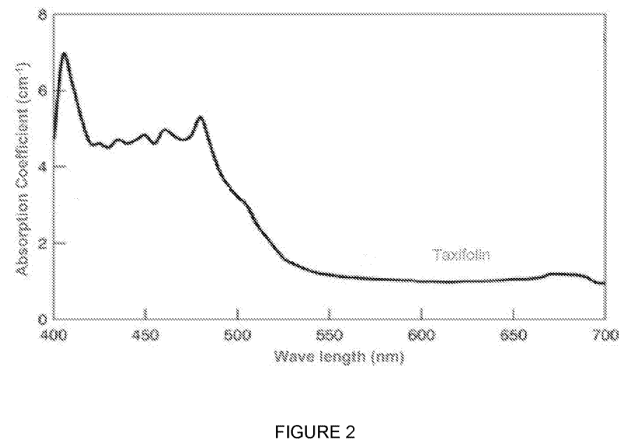

FIG. 2: Absorption spectrum of the base line taxifolin solution (Solution 1:3000 mg Taxifolin/1000 ml) vs air.

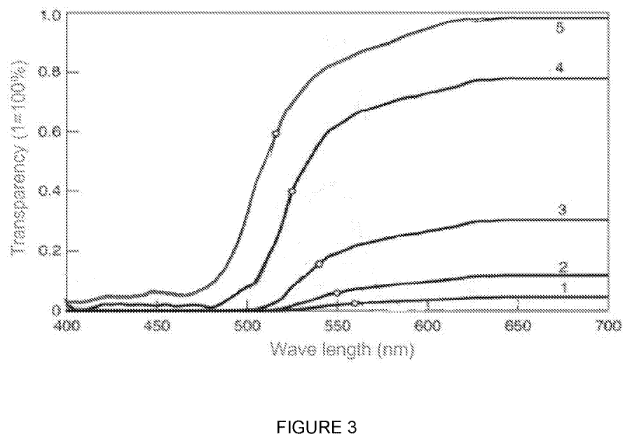

FIG. 3: Taxifolin solution light absorption curves (Solution 1:3000 mg taxifolin/1000 ml; Solution 2:1500 mg taxifolin/1000 ml; Solution 3:750 mg taxifolin/1000 ml; Solution 4:250 mg taxifolin/1000 ml; Solution 5:100 mg taxifolin/1000 ml).

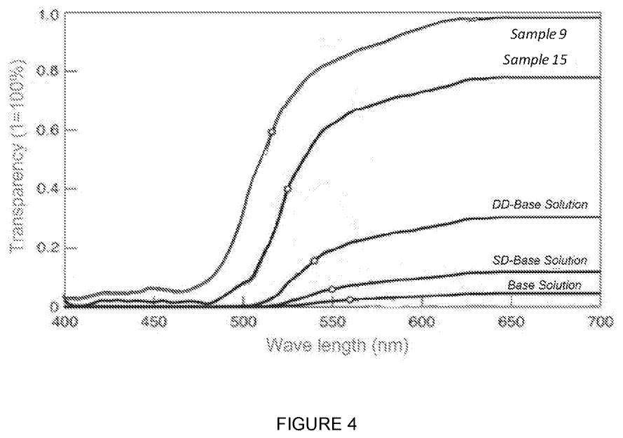

FIG. 4: Light Absorption Curves of Test Solutions of Taxifolin: Base Solution (taxifolin concentration 3000 mg/1000 ml); SD-Base Solution (Single Dilution Base Solution-Taxifolin concentration 1500 mg/1000 ml); DDB (Double Dilution Base Solution-Taxifolin concentration 750 mg/1000 ml); Solution 9 (Taxifolin concentration 0.1 mg/ml) and Solution 15 (Taxifolin concentration 0.25 mg/ml).

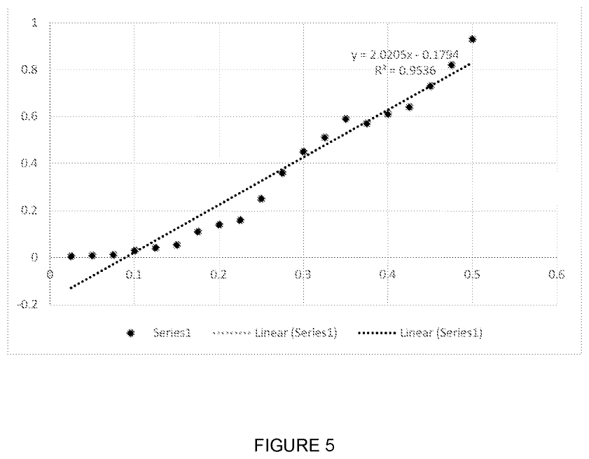

FIG. 5: Linear correlation of different solutions' absorption.

Table 1: Blind Sensorial Perception testing-Taxifolin smell, taste and appearance.

Table 2: Spectroscopic measurements of test samples.

Table 3: Blind Sensorial Perception testing-Taxifolin smell, taste and appearance (additional concentrations).

Table 4: Blind Sensorial Perception testing-Catechin smell, taste and appearance.

Table 5: Blind Sensorial Perception testing-Epicatechin smell, taste and appearance.

Table 6: Blind Sensorial Perception testing-Gallocatechin smell, taste and appearance.

### EXAMPLE 1—WATER APPEARANCE (VISUAL PERCEPTION)

Using taxifolin as an exemplary flavonoid, visual perception was assessed based on two parameters: transparency and colourlessness. In particular, the appearance of water was assessed by measuring the transmission spectrum of various water solutions comprising different concentrations of taxifolin:
Solution 1 (base line): 3000 mg/1000 ml;
Solution 2:1500 mg/1000 ml;
Solution 3:750 mg/1000 ml;
Solution 4:250 mg/1000 ml;
Solution 5:100 mg/1000 ml.

Specially, base line solution (Solution 1) was a commercial dietary supplement ‘Taxifolin Aqua’ (Russia), with the concentration of taxifolin being confirmed as 3000 mg/1000 ml using HPLC (High performance Liquid Chromatography).

Solutions 2-5 were then prepared prepared via dilution of Solution 1 using water purified using MilliQ Academic (Merck Millipore, Germany) with resistance at least 15 K Ohm.
Solution 2:10 ml Solution 1 in 10 ml MilliQ water;
Solution 3:10 ml Solution 2 in 10 ml MilliQ water;
Solution 4:10 ml Solution 3 in 10 ml MilliQ water;
Solution 5:4 ml Solution 4 added to 6 ml water.

Spectrometric measurements were then obtained, using a LOMO SF46 spectrophotometer (Russia) for each of Solutions 1 to 5, the results of which are shown in FIG. 1, FIG. 2 and FIG. 3.

These results show that the tested taxifolin solutions were colourless liquids with an undetectable yellow shade. Therefore, visual appearance of the taxifolin fortified drinking water compositions of the invention will be identical to taxifolin-free water samples.

### Example 2—Taste, Smell and Appearance (Sensorial Perception)

Using taxifolin as an exemplary flavonoid, sensorial perception was assessed based on two parameters: taste and odour. In particular, a trained sensorial panel comprising of 3 males and 8 females age 28-39 were subject to blind testing, rate the test solutions based on (i) taste, using a 5 point scale (where 1 represents a very strong taste and 5 represents exactly the same taste as expected); (ii) odour, using a 10 point scale where 1 represents no odour and 10 represents a very strong odour); and (iii) appearance, using a 10 point scale where 1 represents no colour and 10 indicates a strong coloration.

Two deionised water solutions were tested:
Solution A (control): 100 ml of deionised water;
Solution B (test): 100 ml solution (5.0 mg taxifolin/1000 ml).

The results of the blind testing, which are shown in Table 1, indicate that no difference in taste, odour and/or appearance is discernible between Solutions A and B.

TABLE 1

| Sample | Taste Rating | Odour Rating | Colour Rating |
|---|---|---|---|
| Solution A: | 5 Panelists: 1 | All panelists | All panelists |
| Control | 6 Panelists: 2 | rated 1 | rated 1 |
| Solution B: | 6 Panelists: 1 | All panelists | All panelists |
| Test | 5 Panelists: 2 | rated 1 | rated 1 |

### Example 3—Water Appearance (Visual Perception) at Additional Concentrations

To ensure that addition of taxifolin does not impact aesthetic (visual perception) and sensorial (taste) performance of water, two sets of experiments have been carried out. The first set of experiments dealt with water appearance (transparency/colourless) and the second with water taste. Experiments included taxifolin solutions with concentration ranging from 0.025 mg/ml to 0.5 mg/ml. Visual perception was assessed based on two parameters: transparency and colourlessness.

### Methodology:

The appearance of water was assessed by the transmission spectrum of water solutions with different concentration of taxifolin (dihydroquercetin) ranging from 25 mg in 1000 ml (Solution 6) to 500 mg in 1000 ml (Solution 25). Different samples' concentrations are presented in Table 2. The wavelength of solutions below 25 mg/1000 ml are outside the yellow spectrum absorption range typical for Taxifolin. Thus 25 mg/1000 ml was selected as the lowest test concentration. Spectrometric measurements were done using LOMO SF46 (Russia).

### Solution Preparation:

1) The base solution was a commercial dietary supplement ‘Taxifolin Aqua’ (Russia) and concentration of taxifolin was confirmed using HPLC (High performance Liquid Chromatography): Taxifolin concentration 3000 mg/1000 ml (Base)
2) Two additional base solutions were prepared: single dilution 1500 mg/1000 ml (SDB) and double dilution 750 mg/1000 ml (DDB).
3) The Solutions 6-25 (Table 2) were prepared by diluting corresponding solutions with deionised water purified using MilliQ Academic (Merck Millipore, Germany) with resistance at least 15 K Ohm.

TABLE 2

| | | | Results |
|---|---|---|---|
| | | | Spectrometric |
| | | | Measurements*, |
| | Concentration | | 490 nm**, |
| Solution | Mg/ml | Sample Preparation Dilution | wavelength cm⁻¹, |
| 6 | 0.025 | 10 ml Solution 7 added to 1 ml water | 0.005 |
| 7 | 0.05 | 10 ml Solution 9 added to 10 ml water | 0.008 |
| 8 | 0.075 | 10 ml Solution 11 added to 1 ml water | 0.012 |
| 9 | 0.1 | 10 ml Solution 13 added to 1 ml water | 0.03 |
| 10 | 0.125 | 10 ml Solution 15 added to 10 ml water | 0.041 |
| 11 | 0.15 | 10 ml Solution 17 added to 10 ml water | 0.053 |
| 12 | 0.175 | 10 ml Solution 19 added to 10 ml water | 0.11 |
| 13 | 0.2 | 10 ml Solution 21 added to 10 ml water | 0.14 |
| 14 | 0.225 | 18 ml Solution 15 added to 2 ml water | 0.16 |
| 15 | 0.25 | 10 ml Solution 25 added to 10 ml water | 0.25 |
| 16 | 0.275 | 19 ml Solution 17 added to 1.6 ml ml water | 0.36 |
| 17 | 0.3 | 19 ml Solution 18 added to 2 ml water | 0.45 |
| 18 | 0.325 | 19 ml Solution 19 added to 1 ml water | 0.51 |
| 19 | 0.35 | 19 ml Solution 20 added to 1 ml water | 0.59 |
| 20 | 0.375 | 19 ml Solution 21 added to 1 ml water | 0.57 |
| 21 | 0.4 | 19 ml Solution 22 added to 1 ml water | 0.61 |
| 22 | 0.425 | 19 ml Solution 23 added to 1 ml water | 0.64 |
| 23 | 0.45 | 19 ml Solution 24 added to 1 ml water | 0.73 |
| 24 | 0.475 | 19 ml Solution 25 added to 1 ml water | 0.82 |
| 25 | 0.5 | 10 ml Base solution added in 50 ml water | 0.93 |

*3 repeats per sample
**maximum absorption wavelength of dihydroquercetin in water

The Solutions 6-20 present a colourless liquid with an undetectable yellow shade which started to appear close to Solution 25. Visual appearance of the invention the invention with a concentration 0.25 mg/ml is identical to the original water samples (no Taxifolin).

The linear correlation shows (FIG. 5) that the concentration of taxifolin directly influences the absorbance of a sample. The expectation would be that, as the concentration goes up, more radiation is absorbed and the absorbance goes up. Thus, in the case of linear correlation, the absorbance is directly proportional to the concentration.

In relation to spectroscopic analysis of catechin, epicatechin and gallocatechin, spectroscopic measurements are not applicable as none of the relevant compounds have absorption in the visible range wavelengths. Therefore, the visual appearance of fortified water will not be affected by the addition of any of the abovementioned flavonoids.

### Example 4—Taste, Smell and Appearance (Sensorial Perception) at Additional Concentrations

To determine if there are any sensorial differences between water solution with and without Taxifolin, a study has been performed to asses taste and odour of water solutions.

### Methodology:

Panellists: A trained sensorial panel comprising of 5 males and 8 females age 26-58. Test methodology: Blind testing where panellists were asked to rate the test solutions on (i) taste using 5 point scale where 1 very different taste and 5 exactly the same taste and (ii) odour using 10 point scale where 1 no odour and 10 very strong odour, (iii) appearance using 10 point scale where 1-no colour (colourless) and 10-strong colour.

### Test Solutions:

Control—100 ml of deionised water and Test Solutions-Solutions 6, 11, 16, 21 and 25):
Solution 25 was prepared by adding 50 ml of the base solution to 250 ml of deionised water.
Solution 21 was prepared by addition 40 ml of the base solution to 260 ml of deionised water
Solution 16 was prepared by adding 27.5 ml of the base solution to 272.5 ml of deionised water.
Solution 11 was prepared by adding 15 ml of the base solution to 285 ml of deionised water.
Solution 6 was prepared by adding 2.5 ml of the base solution to 297.5 ml of deionised water.

TABLE 3

| Sample | Taste Rating | Odour Rating | Colour Rating |
|---|---|---|---|
| Control | Panelists 13 rated 1 | All panelists | All panelists |
| Solution | Panelists 0 rated 2-5 | rated 1 | rated 1 1 |
| Solution 25 | Panelists 7 rated 1 | All panelists | All panelists |
| | Panelists 5 rated 2 | rated 1 | rated 1 |
| Solution 21 | Panelists 13 rated 1 | All panelists | All panelists |
| | Panelists 0 rated 2-5 | rated 1 | rated 1 |
| Solution 16 | Panelists 13 rated 1 | All panelists | All panelists |
| | Panelists 0 rated 2-5 | rated 1 | rated 1 |
| Solution 11 | Panelists 13 rated 1 | All panelists | All panelists |
| | Panelists 0 rated 2-5 | rated 1 | rated 1 |
| Solution 6 | Panelists 13 rated 1 | All panelists | All panelists |
| | Panelists 0 rated 2-5 | rated 1 | rated 1 |

All 13 panellists did not see any negative associated with taste, odour and appearance/colour of differences between solutions (Table 3). The only comments were for solution 25 that had a slight bitter tinkle and yellow shade, however, these did not have significant impact the overall product rating. Thus, the data supports that the drinking water of the present disclosure has the same taste as water without Taxifolin.

Experimental data showing that addition of Taxifolin does not impact any aesthetic/sensorial water properties: (1) water appearance (transparency, colour/colourless) and (2) sensorial characterises (no impact on taste, smell and appearance). The present invention has the same properties as water being a tasteless, odourless, and colourless liquid.

### Example 5—Taste, Smell and Appearance (Sensorial Perception)

Using catechin, epicatechin and gallocatechin as further exemplary flavonoids, sensorial perceptions of each of the compounds were assessed based on two parameters: taste and odour.

Test solutions were prepared by dissolving the flavonoids at room temperature (18-21° C.) in purified and deionized water (resistance no less than 15 kOhm, MilliQ Academic purification system).

Sensorial perceptions of fortified water comprising catechin, epicatechin or gallocatechin were evaluated at the following concentrations:
Concentration 1-0.05 mg/ml
Concentration 2-0.1 mg/ml
Concentration 3-0.5 mg/ml
Concentration 4-1.0 mg/ml
Concentration 5-2.0 mg/ml

In particular, a trained sensorial panel were subject to blind testing and sensorial perceptions were assessed using a 10-point scale where 10 corresponded to no differences (colour, taste, odour) vs pure water and 0 to intolerable changes indicating; 8 points—noticeable changes that impact product rating; 6 points—border line changes indicating the emergence of a new descriptor; 4 points—acceptable changes that allow differentiation of the solutions; 2 points—changes in the parameter of the borderline state of attribution to a food product.

The results of the blind testing, which are shown in Tables 4 to 6, indicate that no difference in odour and/or appearance is discernible between fortified water and control (pure) water. Difference in taste only becomes discernible at higher concentrations for catechin and gallocatechin.

TABLE 4

| Catechin | | | |
|---|---|---|---|
| concentration | Sensorial Testing | | |
| mg/ml | Taste Rating | Odour Rating | Colour Rating |
| 0.05 | No taste | No odour | No Colour |
| | (10.0) | (10.0) | (10.0) |
| 0.1 | No taste | No odour | No colour |
| | (10.0) | (10.0) | (10.0) |
| 0.5 | No taste | No odour | No colour |
| | (10.0) | (10.0) | (10.0) |
| 1.0 | Tart | No odour | No colour |
| | (8.0) | (10.0) | (10.0) |
| 2.0 | Tart | No odour | No colour |
| | (6.0) | (10.0) | (10.0) |

TABLE 5

| Epicatechin | | | |
|---|---|---|---|
| concentration | Sensorial Testing | | |
| mg/ml | Taste Rating | Odour Rating | Colour Rating |
| 0.05 | No taste | No odour | No colour |
| | (10.0) | (10.0) | (10.0) |
| 0.1 | No taste | No odour | No colour |
| | (10.0) | (10.0) | (10.0) |
| 0.5 | No taste | No odour | No colour |
| | (10.0) | (10.0) | (10.0) |
| 1.0 | No taste | No odour | No colour |
| | (10.0) | (10.0) | (10.0) |
| 2.0 | No taste | No odour | No colour |
| | (10.0) | (10.0) | (10.0) |

TABLE 6

| Gallocatechin | | | |
|---|---|---|---|
| concentration | Sensorial Testing | | |
| mg/ml | Taste Rating | Odour Rating | Colour Rating |
| 0.05 | No taste | No odour | No colour |
| | (10.0) | (10.0) | (10.0) |
| 0.1 | No taste | No odour | No colour |
| | (10.0) | (10.0) | (10.0) |
| 0.5 | No taste | No odour | No colour |
| | (10.0) | (10.0) | (10.0) |
| 1.0 | Light biter | No odour | No colour |
| | (8.0) | (10.0) | (10.0) |
| 2.0 | Bitter | No odour | No colour |
| | (6.0) | (10.0) | (10.0) |

### REFERENCES

1 Karatoprak G S, Antioxidants with hepatoprotective activity. in Influence of Nutrients, Bioactive Compounds, and Plant Extracts in Liver Diseases, 2021
2 Dissecting Efficacy and Metabolic Characteristic Mechanism of Taxifolin on Renal Fibrosis by Multivariate Approach and Ultra-Performance Liquid Chromatography Coupled With Mass Spectrometry-Based Metabolomics Strategy
By: Ren, Lei; Guo, Hao-Nan; Yang, Jun; et al.
FRONTIERS IN PHARMACOLOGY Volume: 11 Article Number: 608511 Published: Jan. 14, 2021
3 Protective effects of taxifolin on pazopanib-induced liver toxicity: an experimental rat model
By: Akagunduz, Baran; Ozer, Muhammet; Ozcicek, Fatih; et al.
EXPERIMENTAL ANIMALS Volume: 70 Issue: 2 Pages: 169-176 Published: 2021
4 Taxifolin improves disorders of glucose metabolism and water-salt metabolism in kidney via PI3K/AKT signaling pathway in metabolic syndrome rats
By: Gao, Liyuan; Yuan, Peipei; Zhang, Qi; et al.
LIFE SCIENCES Volume: 263 Article Number: 118713 Published: Dec. 15, 2020
5 Anti-hyperuricemic effect of taxifolin in cultured hepatocytes and model mice
By: Adachi, Shin-ichi; Nihei, Ken-ichi; Ishihara, Yoshiyuki; et al.
CYTOTECHNOLOGY Volume: 69 Issue: 2 Pages: 329-336 Published: April 2017
6 Mechanisms Modified by (−)-Epicatechin and Taxifolin Relevant for the Treatment of Hypertension and Viral Infection: Knowledge from Preclinical Studies
By: Bernatova, Iveta; Liskova, Silvia
ANTIOXIDANTS Volume: 10 Issue: 3 Article Number: 467 Published: March 2021
Dietary Taxifolin Protects Against Dextran Sulfate Sodium-Induced Colitis via NF-kappa B Signaling, Enhancing Intestinal Barrier and Modulating Gut Microbiota
By: Hou, Jinxiu; Hu, Mingyang; Zhang, Le; et al.
FRONTIERS IN IMMUNOLOGY Volume: 11 Article Number: 631809 Published: Feb. 16, 2021
7 Taxifolin inhibits amyloid-beta oligomer formation and fully restores vascular integrity and memory in cerebral amyloid angiopathy
By: Saito, S.; Yamamoto, Y.; Maki, T.; et al.
Conference: 23rd World Congress of Neurology (WCN) Location: Kyoto, JAPAN Date: September 2017 16-21, Sponsor(s): World Federat Neurol
JOURNAL OF THE NEUROLOGICAL SCIENCES Volume: 381 Supplement: S Pages: 988-988 Meeting Abstract: 2752 Published: Oct. 15, 2017
8 Taxifolin inhibits amyloid-beta oligomer formation and fully restores vascular integrity and memory in cerebral amyloid angiopathy
By: Saito, Satoshi; Yamamoto, Yumi; Maki, Takakuni; et al.
ACTA NEUROPATHOLOGICA COMMUNICATIONS Volume: 5 Article Number: 26 Published: Apr. 4, 2017
9. Taxifolin protects against cardiac hypertrophy and fibrosis during biomechanical stress of pressure overload
By: Guo, Haipeng; Zhang, Xin; Cui, Yuqian; et al.

### TOXICOLOGY AND APPLIED PHARMACOLOGY Volume: 287 Issue: 2 Pages: 168-177 Published: Sep. 1, 2015

10 Efficacy of (+/−)-taxifolin from Larix sibirica (Munchh.) Ledeb. on blood pressure in experiments in vivo
By: Tikhonov, V. P.; Makarova, M. N.; Zajtseva, M. A.; et al.
Conference: 54th Annual Congress on Medicinal Plant Research Location: Helsinki, FINLAND Date: Aug. 29-Sep. 2, 2006 Sponsor(s): Soc Medicinal Plant Res; Univ Helsinki, Fac Pharm
PLANTA MEDICA Volume: 72 Issue: 11 Pages: 1035-1035 Meeting Abstract: P174 Published: September 2006
11 EFFECTS OF TAXIFOLIN ON THE SERUM-CHOLESTEROL LEVEL IN RATS
By: ITAYA, S; IGARASHI, K
BIOSCIENCE BIOTECHNOLOGY AND BIOCHEMISTRY Volume: 56 Issue: 9 Pages: 1492-1494 Published: September 1992
12 Taxifolin and Vitamin C and Vitamin E: antioxidant synergetic action
Quercetin and Vitamin C: An Experimental, Synergistic Therapy for the Prevention and Treatment of SARS-COV-2 Related Disease (COVID-19) Immunol., 19 Jun. 2020|https://doi.org/10.3389/fimmu.2020.01451
13 Vitamin C and Dihydroquercetin: Few adults realize how quickly the body oxidizes and excretes vitamin C. Here we introduce a powerful new antioxidant that helps maintain and re-circulate vitamin C in the body, thus augmenting the disease-fighting capabilities of this essential nutrient.
Scientifically reviewed by: Dr. Gary Gonzalez, MD, on January 2021. Written By Mark J. Neveu
https://www.lifeextension.com/magazine/2006/12/report_vitaminc
14 https://www.nutranews.org/en-veinotonics-taxifolin-an-important-role-in-capillary-health-1288
Antioxidant activity of taxifolin derived from larch: synergistic studies
Bulgarian Chemical Communications, Volume 51, Special Issue A (pp. 172-176) 2019
http://www.bcc.bas.bg/BCC_Volumes/Volume_51_Special_A_2019/B-pdf-ready/BCC-51-A-2019-172-176-Galunska-A9.pdf
15 Methodical recommendations MP 2.3.1.1915-04 “Recommended levels of consumption of food and biologically active substances” (approved by the Russian Federal Service for Supervision of Consumer Rights Protection and Human Welfare on Jul. 2, 2004
16 DHQ in Soft Drinks & Alcoholic Beverages Industries' post on BALINVEST website, dated 11 Dec. 2017
https://www.balinvest.lv/applications-of-dhq/blog-5
17 Gratton G, Weaver S R, Burley C V, Low K A, Maclin E L, Johns P W, Pham Q S, Lucas S J, Fabiani M, Rendeiro C. Dietary flavanols improve cerebral cortical oxygenation and cognition in healthy adults. Scientific reports. 2020 Nov. 24; 10 (1): 1-3.

1. A drinking water composition having a pH of from 4 to 9.5, wherein said composition is fortified with one or more flavonoid compound in a total amount of at least 0.025 mg/ml, wherein said composition contains no additional natural or artificial colorants, flavours or preservatives.

2. The composition according to claim 1, wherein said one or more flavonoid compound is selected from the group consisting of: flavan-3-ols (also known as flavanols), anthocyanidins, flavanones, flavones, flavonols, flavanonols, and/or glycosides thereof.

3. The composition according to claim 1, wherein said one or more flavonoid compound is selected from the group consisting of: flavan-3-ols (also known as flavanols), flavonols, flavanonols, and/or glycosides thereof.

4. The composition according to claim 1, wherein said flavonoid compound is selected from taxifolin, catechin, epicatechin and/or gallocatechin.

5. The composition according to claim 1, wherein said flavonoid compound is selected from taxifolin.

6. The composition according to claim 1, wherein said flavonoid compound is selected from catechin, epicatechin and/or gallocatechin

7. The composition according to claim 1, comprising said or more flavonoid compound in an amount from 0.025 mg/ml to 0.5 mg/ml.

8. The composition according to claim 7, comprising said flavonoid compound(s) in a total amount of about 0.05 mg/ml or more.

9. The composition according to claim 1, wherein said one or more flavonoid compound is provided as a stabilized water soluble flavonoid solution.

10. The composition according to claim 9, wherein said stabilized water soluble flavonoid solution is a stabilized water soluble taxifolin solution.

11. The composition according to claim 10, wherein said water soluble taxifolin solution comprises taxifolin; 2-hydroxypropyl-beta-cyclodextrin; one or more of propylene glycol, glycerol and PEG 400; one or more organic acid or a water-soluble salt thereof; and one or more carbohydrates selected from monosaccharides, disaccharides and maltodextrins in the following amounts, with distilled water to balance:
Taxifolin: 0.1 to 30 wt. %
2-hydroxypropyl-beta-cyclodextrin: 0.01-25 wt. %
propylene glycol, glycerin and/or PEG-400:1-70 wt. %
organic acid/salt: 0.1-3 wt. %
carbohydrate: 0.01-30 wt. %

12. The composition according to claim 11, wherein:
a. said organic acid is selected from the group consisting of succinic acid, malic acid, ascorbic acid, isoascorbic acid, citric acid, lactic acid, fumaric acid, tartaric acid, acetic acid, sorbic acid, benzoic acid, and combinations thereof; and/or
b. said carbohydrate is selected from the group consisting of glucose, fructose, sorbitol, xylitol, galactose, mannose, sucrose, lactose, maltose, isomaltose, lactulose, and combinations thereof.

13. The composition according to claim 1, further comprising one or more additive selected from vitamin C and vitamin E.

14. The composition according to claim 1, wherein said water is mineral water.

15. The composition according to claim 14, wherein said mineral water is spring water or well water.

16. A packaged drinking water product comprising a composition according to claim 1 disposed within an oxygen impermeable container.

17. The packaged drinking water product according to claim 16, wherein said container is in the form of a can or a bottle.

18. A method of preparing a packaged drinking water product according to claim 16, said method comprising the following steps:
a. adding one or more flavonoid compounds and, optionally, any further additives to drinking water to prepare a flavonoid fortified drinking water composition;
b. filling one or more oxygen impermeable container with said flavonoid fortified drinking water composition; and
c. sealing said one or more oxygen impermeable container.

19. The method according to claim 18, further comprising purifying and/or sterilizing the flavonoid fortified drinking water composition prior to commencing step c).

20. The method according to claim 19, wherein said purifying and/or sterilizing comprises one or more processes selected from disinfection, filtration, decanting, CO2 removal, and activated alumina treatment.
